(19) United States
(12) Patent Application Publication
(10) Pub. No.: US 2007/0155832 A1
(43) Pub. Date: Jul. 5, 2007

(54) Use of sulfonamide compounds for the treatment of diabetes and/or obesity

(76) Inventors: Samantha Louise Budd Haeberlein, Sodertalje (SE); Linda Karen Buckett, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 10/577,357

(22) PCT Filed: Oct. 28, 2004
(86) PCT No.: PCT/GB04/04582

§ 371 Date: Apr. 28, 2006

(30) Foreign Application Priority Data
Oct. 29, 2003 (GB) 0325192.3

(51) Int. Cl. A61K 31/325; A61K 31/255; A61K 31/17

(52) U.S. Cl. 514/476; 514/594; 514/517

(57) ABSTRACT

The use of a compound of Formula (1): wherein for example: X and Y are independently selected from: oxygen, sulphur and (—CR^{a}R^{b}—)ₙ; wherein: n is an integer of from 1 to 4 and R^{a} and R^{b} are each independently selected from hydrogen, C₁₋₆altkyl, C₁₋₆alkoxy, halo, hydroxy, C₁₋₆alkanoyloxy, C₃₋₁₂cycloalkyl and optionally substituted phenyl or R^{a} and R^{b} together form a C₅₋₁₂spirocycloalkyl or a carbonyl; R¹ and R³ are independently selected from (a) optionally substituted phenyl or phenoxy; (b) optionally substituted naphth-1-yl or naphth-2-yl; (c) arylC₁₋₆alkyl; (d) C₁₋₂₀alkyl or C₁₋₂₀alkenyl; and (e) adamantyl or a C₃₋₁₂cycloalkyl R² is hydrogen, a C₁₋₈alkyl or benzyl; or pharmaceutically acceptable salt, pro-drug or solvate thereof as DGAT inhibitors and in the manufacture of a medicament for the treatment of type II diabetes and/or obesity.

The present invention relates to a novel use of sulphonamide compounds of Formula (I) as inhibitors of both acetyl CoA(acetyl coenzyme A):diacylglycerol acyltransferase and acetyl CoA:cholesterol acyl transferase and to their use in the treatment of type II diabetes, insulin resistance, impaired glucose tolerance and obesity.

A key enzyme in triglyceride synthesis is acyl CoA:diacylglycerol acyltransferase (DGAT), which is found in the microsomal fraction of cells. DGAT catalyzes the final reaction in the glycerol phosphate pathway, considered to be the main pathway of triglyceride synthesis in cells. The enzyme is also believed to catalyze the final step of the monoacylglycerol pathway, found predominantly in enterocytes of the small intestine. In both pathways, DGAT facilitates the joining of a diacylglycerol with a fatty acyl CoA, resulting in the formation of triglyceride. Although it is unclear whether DGAT is rate-limiting for triglyceride synthesis, it catalyzes the only step in the pathway that is committed to producing this type of molecule [Lehner & Kuksis (1996) Biosynthesis of triacylglycerols. Prog. Lipid Res. 35: 169-201].

In 1998, a DGAT gene was identified from sequence database searches because of its similarity to acyl CoA:cholesterol acyltransferase (ACAT) genes. [Cases et al (1998) Identification of a gene encoding an acyl CoA:diacylglycerol acyltransferase, a key enzyme in triacylglycerol synthesis. Proc. Natl. Acad. Sci. USA 95: 13018-13023]. DGAT activity has been found in many mammalian tissues, including rat adipocytes, differentiated 3T3-L1 adipocytes, enterocytes of the small intestine and mammary gland. DGAT is active in both skeletal and heart muscle, where triglycerides serve as stores of fatty acids for oxidative. metabolism.

Because of the previous lack of molecular probes, little is known about the regulation of DGAT. DGAT is known to be significantly up-regulated during adipocyte differentiation. As DGAT acts at an important branch point in the glycerolipid synthetic pathway, its activity may also be regulated in accordance with the metabolic state of the cell. Several studies have reported that hormones influence DGAT activity and the enzyme may be post-translationally regulated by a tyrosine kinase.

Studies in gene knockout mice has indicated that modulators of the activity of DGAT would be of value in the treatment of type II diabetes and obesity. DGAT knockout (Dgat^{-/-}) mice, are viable and capable of synthesizing triglycerides, as evidenced by normal fasting serum triglyceride levels and normal adipose tissue composition. Dgat^{-/-}mice have less adipose tissue than wild-type mice at baseline and are resistant to diet-induced obesity. This is not due to decreased caloric intake in these animals. However, metabolic rate is ˜20% higher in Dgat^{-/-}mice than in wild-type mice on both regular and high-fat diets [Smith et al (2000) Obesity resistance and multiple mechanisms of triglyceride synthesis in mice lacking DGAT. Nature Genetics 25: 87-90]. Increased physical activity in Dgat^{-/-}mice partially accounts for their increased energy expenditure. The Dgat^{-/-}mice also exhibit increased insulin sensitivity and a 20% increase in glucose disposal rate. Leptin levels are 50% decreased in the Dgat^{-/-}mice in line with the 50% decrease in fat mass.

When Dgat^{-/-}mice are crossed with oblob mice, these mice exhibit the ob/ob phenotype [Chen et al (2002) Increased insulin and leptin sensitivity in mice lacking acyl CoA:diacylglycerol acyltransferase J. Clin. Invest. 109:1049-1055]. When Dgat^{-/-}mice are crossed with Agouti mice a decrease in body weight is seen with normal glucose levels and 70% reduced insulin levels compared to wild type, agouti or ob/obl Dgat^{-/-}mice. Food intake is the same but activity is increased. Expression changes in brown and white adipose tissue are consistent with activation of the leptin pathway. These changes are absent in ob/ob mice.

Transplantation of adipose tissue from Dgat^{-/-}mice to wild type mice confers resistance to diet-induced obesity and improved glucose metabolism in these mice [Chen et al (2003) Obesity resistance and enhanced glucose metabolism in mice transplanted with white adipose tissue lacking acyl CoA:diacylglycerol acyltransferase J. Clin. Invest. 111: 1715-1722].

Recently a DGAT enzyme without sequence homology to previously identified DGAT genes was isolated from the fungus Mortierella rammaiziana. A mammalian gene family related to this fungal DGAT has been identified. One member of this family, DGAT2, has been cloned and characterised [Cases et al (2001) Cloning of DGAT2, a second mammalian diacylglycerol acyltransferase, and related family members. J. Biol. Chem. 276:38870-38876.]. DGAT2 has no sequence homology with DGAT1 but shares some homology with the monoacylglycerol acyltransferase (MGAT) family [Yen et al (2002) Identification of a gene encoding MGAT1, a monoacylglycerol acyltransferase. Proc. Natl. Acad. Sci. USA 30 99:8512-8517]. The two DGATs exhibit different sensitivities to MgCl₂. Over-expression of DGAT2 in insect cells results in large increases in triglyceride synthesis from oleoyl CoA and diacylglycerol. DGAT1 and 2 have similar maximal capacities for triglyceride synthesis and have similar fatty acyl CoA specificities. The relative contribution of the various DGATs to triglyceride synthesis in adipose and other tissues remains to be determined although the residual activity in Dgat^{-/-}tissues (DGAT1³¹ /⁻) is relatively low even when low MgCl₂ concentrations are used. Recent evidence suggests that the overt DGAT activity found in hepatocytes is associated with DGAT2.

Acyl-CoA:cholesterol acyltransferase (ACAT) enzymes catalyze the synthesis of cholesterol esters from free cholesterol and fatty acyl-CoAs thereby participating in regulating the concentration of cellular free sterols. The cholesterol ester products of ACAT reactions can be stored in cytosolic droplets, which may serve to protect cells from the toxicity of free cholesterol. In macrophages, the accumulation of these droplets results in the formation of ‘foam cells’, a hallmark of early atherosclerotic lesions [Brown & Goldstein (1983) Annu. Rev. Biochem. 52:223-261.]. In hepatocytes and enterocytes, cholesterol esters can be incorporated into apolipoprotein B-containing lipoproteins for secretion from the cell and hence ACAT enzymes play key regulatory roles in intestinal cholesterol absorption and in hepatic synthesis and secretion of lipoproteins.

In 1993, Chang and co-workers succeeded in cloning a human ACAT gene, now known as ACAT1 [Chang et al (1993) Molecular cloning and functional expression of human acyl-coenzyme A:cholesterol acyltransferase cDNA in mutant Chinese hamster ovary cells. J. Biol. Chem. 268: 20747-20755]. The cloning of ACAT1 led to the identification of a second ACAT gene, ACAT2 [Anderson et al.(1998) Identification of a form of acyl-CoA:cholesterol acyltransferase specific to liver and intestine in nonhuman primates. J. Biol. Chem. 273: 26747-26754; Cases et al.(1998) ACAT-2, a second mammalian acyl-CoA:cholesterol acyltransferase. Its cloning, expression, and characterisation. J. Biol. Chem. 273: 26755-26764; Oelkers et al (1998) Characterisation of two human genes encoding acyl coenzyme A:cholesterol acyltransferase-related enzymes. J. Biol. Chem. 273: 26765-26771]. ACAT1 has many hydrophobic regions, consistent with it being an integral membrane protein. Most of the seven transmembrane domains have sequences that are highly conserved among other ACAT enzymes. As these regions are not conserved in DGAT enzymes, it has been hypothesized that these regions bind cholesterol in the membrane.

ACAT1 mRNA is expressed ubiquitously in mammalian tissues. Expression levels of ACAT1 are highest in the adrenal glands, macrophages, and sebaceous glands; and in humans is also detectable in liver and intestinal epithelial cells. ACAT1 expression has also been detected in human atherosclerotic lesions.

ACAT1 appears to be regulated primarily by posttranslational mechanisms and is allostexically activated by the binding of cholesterol or oxysterols.

The ACAT2 cDNA encodes a protein with greater than 40% identity to human ACAT1. ACAT2 is also a hydrophobic protein with multiple transmembrane domains. ACAT activity is found primarily in the endoplasmic reticulum. The active site for ACAT2 is located on the luminal side of the endoplasmic reticulum membrane, whereas the active site for ACAT1 is oriented toward the cytosol. The ACAT2 sequence contains many of the same motifs found in ACAT1.

ACAT2 is expressed primarily in the liver and small intestine. In humans, nonhuman primates and mice, ACAT2 appears to be the major ACAT in the small intestine. ACAT2 also appears to be the predominant ACAT expressed in the liver of adult nonhuman primates and mice.

International patent application, publication number, WO 94/26702, describes a group of sulphonamide compounds, as ACAT inhibitors, with utility in the treatment of hypercholesterolemia and atherosclerosis. We have surprisingly found that these compounds are also inhibitors of DGAT and thus of utility in the treatment of type II diabetes and obesity.

Thus, according to the first aspect of the invention there is provided the use of a compound of Formula (I) 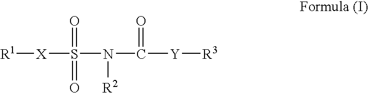
wherein
X and Y are independently selected from: oxygen, sulphur and (—CR^{a}R^{b}—)ₙ; wherein:
   n is an integer of from 1 to 4 and
   R^{a} and R_{b} mare each independently selected from hydrogen, C₁₋₆aLkyl, C₁₋₆alkoxy, halo, hydroxy, C₁₋₆alkanoyloxy, C₃₋₁₂cycloalkyl and optionally substituted phenyl or R^{a} and R^{b} together form a C₅₋₁₂spirocycloalkyl or a carbonyl;
      with the proviso that at least one of X and Y is (—CR^{a}R^{b}—)ₙ and with the further proviso that when X and Y are both (—CR^{a}R^{b}—)ₙ and R^{a} and R^{b} are hydrogen and n is 1, then R¹ and R³ are both aryl;
R¹ and R³ are independently selected from
(a) phenyl or phenoxy wherein the phenyl or phenoxy group is optionally substituted with 1 to 5 substituents independently selected from phenyl, C₁₋₆alkyl, C₁₋₆alkoxy, phenoxy, hydroxy, fluorine, chlorine, bromine, nitro, trifluoromethyl, carboxy, C₁₋₄alkoxycarbony and —(CH₂)ₚNR₄R₅ wherein p is 0 or 1, and R⁴ and R⁵ are independently selected from hydrogen or C₁₋₄alkyl;
(b) naphth-1-yl or naphth-2-yl wherein the naphthyl group is optionally substituted with from 1 to 3 substituents independently selected from phenyl, C₁₋₆alkyl, C₁₋₆alkoxy, phenoxy, hydroxy, fluorine, chlorine, bromine, nitro, trifluoromethyl, carboxy, C₁₋₄alkoxycarbony and —(CH₂)ₚNR₄R₅ wherein p, R⁴ and R⁵ are as defined above;
(c) arylC₁₋₆alkyl;
(d) C₁₋₂₀alkyl or C₁₋₂₀alkenyl; and
(e) adamantyl or a C₃₋₁₂cycloalkyl;
R² is hydrogen, a C₁₋₈alkyl or benzyl;
   or pharmaceutically acceptable salt, pro-drug or solvate thereof in the manufacture of a medicament for the treatment of type II diabetes and/or obesity.

Preferably only if X is (—CR^{a}R^{b}—), can R¹ be optionally substituted phenoxy, and only if Y is (—CR^{a}R^{b}—)ₙ can R³ be optionally substituted phenoxy.

According a further feature of the first aspect of the invention there is provided a method of treatment, in a warm-blooded animal, of type II diabetes and/or obesity comprising the administration of a therapeutically (including prophylactically) effective amount of a compound of formula (I) or a pharmaceutically acceptable salt, pro-drug or solvate thereof.

According to a further feature of the first aspect of the invention there is provided a pharmaceutical composition comprising a compound of Formula (I), or a pharmaceutically effective salt, pro-drug or solvate thereof, in admixture with a pharmaceutically acceptable diluent or carrier for the treatment of type II diabetes and/or obesity.

According to a further feature of the first aspect of the invention there is provided the use of a compound of Formula (I) in the manufacture of a medicament for the inhibition of both acetyl CoA(acetyl coenzyme A):diacylglycerol acyltransferase and acetyl CoA:cholesterol acyl transferase.

According a further feature of the first aspect of the invention there is provided a method of treatment, in a warm-blooded animal, by the inhibition of both acetyl CoA(acetyl coenzyme A):diacylglycerol acyltransferase and acetyl CoA:cholesterol acyl transferase comprising the administration of a therapeutically (including prophylactically) effective amount of a compound of Formula (I) or a pharmaceutically acceptable salt, pro-drug or solvate thereof.

According to a further feature of the first aspect of the invention there is provided a pharmaceutical composition comprising a compound of Formula (I), or a pharmaceutically effective salt, pro-drug or solvate thereof, in admixture with a pharmaceutically acceptable diluent or carrier for the inhibition of both acetyl CoA(acetyl coenzyme A):diacylglycerol acyltransferase and acetyl CoA:cholesterol acyl transferase.

In the present specification, unless otherwise indicated, an alkyl group is a saturated chain having 1 to 20 carbon atoms which may be linear or branched. Examples of alkyl groups include: methyl, ethyl, n-propyl, isopropyl, n-butyl, iso-butyl, tert-butyl, n-pentyl, isopentyl, n-hexyl, n-heptyl, n-octyl, n-undecyl, n-dodecyl, n-hexadecyl, 2,2-dimethyldodecyl, 2-tetradecyl, and n-octadecyl groups.

The term “alkenyl” refers to a carbon chain having 1 to 20 carbon atoms having from 1 to 3 double bonds. Examples of alkenyl include: ethenyl, 2-propenyl, 2-butenyl, 3-pentenyl, 2-octenyl, 5-nonenyl, 4-undecenyl, 5-heptadecenyl, 3-octadecenyl, 9-octadecenyl, 2,2-dimethyl-11-eicosenyl, 9,12-octadecadienyl, and hexadecenyl.

The term “aryl” refers to phenyl or naphthyl.

The term “halo” refers to fluoro, chloro, bromo or iodo.

The term “cycloalkyl” refers to a saturated carbocyclic ring containing between 3 and 12 carbon atoms, preferably between 3 and 8 carbon atoms. Examples of cycloalkyl include: cyclopentyl, cyclohexyl, cyclooctyl, tetrahydronaphthyl, adamant-1-yl and adamant-2-yl.

The term “spirocycloalkyl” refers to bicyclic saturated carbon rings containing between 5 and 12 carbon atoms wherein one carbon atom is common to both rings, Examples of spirocycloalkyl include: spirocyclopropyl, spirocyclobutyl, spirocyclopentyl and spirocyclohexyl.

The term “treatment” refers to both treatment and prevention.

Examples of C₁₋₈alkoxy include methoxy, ethoxy, n-propoxy, t-butoxy, and pentyloxy; examples of C₁₋₆alkanoyl incude formyl, ethanoyl, propanoyl or pentanoyl, examples of arylC₁₋₆alkyl include benzyl, phenethyl, 3-phenylpropyl, 2-phenylpropyl, 4-phenylbutyl, 2-phenylbutyl, 3-phenylbutyl, benzhydryl, 2,2-diphenylethyl and 3,3-diphenylbutyl.

It is to be understood that, insofar as certain of the compounds of the invention may exist in optically active or racemic forms by virtue of one or more asymmetric carbon atoms, the invention includes in its definition any such optically active or racemic form which possesses the property of treating type II diabetes and/or obesity. The synthesis of optically active forms may be carried out by standard techniques of organic chemistry well known in the art, for example by synthesis from optically active starting materials or by resolution of a racemic form. Similarly, activity of these compounds may be evaluated using the standard laboratory techniques referred to hereinafter.

The invention also relates to any and all tautomeric forms of the compounds of the different features of the invention that possess the property of treating type II diabetes and/or obesity.

It will also be understood that certain compounds of the present invention may exist in solvated, for example hydrated, as well as unsolvated forms. It is to be understood that the present invention encompasses all such solvated forms which possess the property of treating type II diabetes and/or obesity.

Preferred compounds of Formula (I) are those wherein any one of the following apply:
(i) R¹ and R³ are selected from either of the following:
   (a) R¹ is phenyl or phenyl disubstituted in the 2,6-positions and R³ is phenyl or is phenyl disubstituted in the 2,6-positions;
   (b) each of R¹ and R³ is phenyl disubstituted in the 2,6-position,
   (c) R¹ is phenyl disubstituted in the 2,6-positions and R³ is phenyl trisubstituted in the 2,4,6-positions;
   (d) R¹ is 2,6-bis(1-methylethyl)phenyl and R³ is 2,6-bis(l-methylethyl)phenyl or 2,4,6-tris(1-methylelthyl)phenyl,
   (e) one of R¹ and R³ is the group 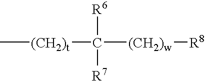
   wherein
      t is 0to 4;
      w is 0 to 4 with the proviso that the sum of t and w is not greater than 5;
      R⁶ and R⁷ are independently selected from hydrogen and C₁₋₆alyl, or when R⁶ is hydrogen, R⁷ can be selected from the groups defined for R⁸ ; and R⁸ is phenyl optionally substituted with from 1 to 3 substituents selected C₁₋₆alkyl C₁₋₆alkoxy, phenoxy, hydroxy, fluorine, chlorine, bromine, nitro, trifluoromethyl, carboxy, C₁₋₄alkoxycarbonyl, and —(CH₂)pNR⁴R⁵ wherein p, R⁴ and R⁵ are as defined above.
(ii) one of R¹ and R³ is phenyl, and more preferably wherein one of R¹ and R³ is substituted phenyl, and still more preferably wherein one of R¹ and R³ is phenyl disubstituted in the 2,6-positions.
(iii) both R¹ and R³ are phenyl disubstituted in the 2,6-positions.
(iv) R¹ is phenyl disubstituted in the 2,6-position and R³ is trisubstituted in the 2,4,6 -positions.
(v) R¹ is 2,6-bis(1-methylethyl)phenyl and R³ is 2,6-bis(l-methylethyl)phenyl or 2,4,6-tris(1-methylethyl)phenyl.

According to a further feature of the invention there is provided the use of the following preferred group of compounds, or pharmaceutically-acceptable salt, pro-drug or solvate thereof:
(I) X is selected from oxygen, sulfur and (—CR^{a}R^{b}—)ₙ;
   Y is selected from oxygen, sulfur and (—CR^{a}R^{b}—)ₙ, with the proviso that at least one of X or Y is (—CR^{a}R^{b}—)ₙ wherein n is an integer of from 1 to 4 and R^{a} and R^{b} are independently selected from hydrogen, C₁₋₆alkyl, optionally substituted phenyl, halo, hydroxy, C₁₋₆alkoxy, C₁₋₆alkanoyloxy and C₃₋₁₂cycloalkyl, or R^{a} and R^{b} taken together form a carbonyl or C₃₋₁₀spirocycloalkyl;
   R¹ is selected from optionally substituted phenyl, C₁₋₁₀alkyl and C₃₋₁₀cycloalkyl;
   R² is hydrogen;
   R³ is selected from optionally substituted phenyl, C₁₋₁₀alkyl, C₃₋₈cycloalkyl, optionally substituted phenoxy; with the proviso that only if X is (—CR^{a}R^{b}—)ₙ can R¹ be optionally substituted phenoxy and only if Y is (—CR^{a}R^{b}—)ₙ can R³ be optionally substituted phenoxy, and with the further proviso that at least one of R¹ and R³ is optionally substituted phenyl or phenoxy.
(II) X is oxygen;
   Y is (—CR^{a}R^{b}—)ₙ wherein n is an integer of from 1 to 2;
   R¹ is optionally substituted phenyl;
   R² is hydrogen;
   R³ is selected from optionally substituted phenyl, optionally substituted phenoxy, C₁₋₁₀alkyl and C₃₋₁₀cycloalkyl; and
   R^{a} and R^{b} are independently selected from hydrogen, C₁₋₆alkyl, optionally substituted phenyl, halo, hydroxy, C₁₋₆alkoxy, C₁₋₆alkanoyloxy, C₃₋₁₂cycloalkyl, or R^{a} and R^{b} taken together form a carbonyl or a C₅₋₁₂spirocycloalkyl.
(III) X is oxygen;
   Y is (—CR^{a}R^{b}—)n wherein n is an integer of from 1 to 4 and R′ and R″ are each independently hydrogen, alkyl, alkoxy, halogen, hydroxy, acyloxy, cycloalkyl, phenyl optionally substituted or R′ and R″ together form a spirocycloalkyl or a carbonyl;
   R¹ and R³ are independently selected from
   (a) phenyl or phenoxy wherein the phenyl or phenoxy group is optionally substituted with 1 to 5 substituents independently selected from phenyl, C₁₋₆alkyl, C₁₋₆alkoxy, phenoxy, hydroxy, fluorine, chlorine, bromine, nitro, trifluoromethyl, carboxy, C₁₋₄alkoxycarbony and —(CH₂)ₚNR₄R₅ wherein p is 0 or 1, and R⁴ and R⁵ are independently selected from hydrogen or C₁₋₄alkyl;
   (b) naphth-1-yl or naphth-2-yl wherein the naphthyl group is optionally substituted with from 1 to 3 substituents independently selected from phenyl, C₁₋₆alkyl, C₁₋₆alkoxy, phenoxy, hydroxy, fluorine, chlorine, bromine, nitro, trifluoromethyl, carboxy, C₁₋₄alkoxycarbony and—(CH₂)ₚNR₄R₅ wherein p, R⁴ and R⁵ are as defined above;
   (c) arylC₁₋₆alkyl;
   (d) C₁₋₂₀alkyl or C₁₋₂₀alkenyl; and
   (e) adamantyl or a C₃₋₁₂cycloalkyl
R² is hydrogen, a C₁₋₈alkyl or benzyl.

Particularly preferred compounds for use in the method according to the present invention are wherein the compound is selected from:
Sulfamic acid (phenylacetyl)-2,6-bis(1-methylethyl)phenyl ester,
Sulfamic acid [[2,4,6-tris(1-methylethyl)phenyl]acetyl-2,6-bis(1-methylethyl)phenyl ester,
Sulfamic acid[[2,6-bis(1-methylethyl)phenyl]acetyl]-2,6-bis(1-methylethyl)phenyl ester,
Sulfarnic acid [[2,4,6-tris(1-methylethyl)phenyl]acetyl-2,4,6-tris(1-methylethyl)phenyl ester,
Sulfamic acid [[2,6-bis(1-methylethyl)phenyl]acetyl]-2,4,6-tris(1-methylethyl)phenyl ester,
Sulfamic acid[adamantaneacetyl]-2,6-bis[1-methylethyl)phenyl ester
Sulfamic acid[[2,6-bis(1-methylethyl)phenyl]acetyl]-2,6-bis(1-methylethyl)phenyl ester-sodium salt,
Sulfamic acid[[2,4,6-tris(1-methylethyl)phenyl]acetyl]-2,6-bis(1-methylethyl)phenyl ester-sodium salt,
Sulfamic acid (decanoyl)-2,6-bis-(1-methylethyl)phenyl ester,
Sulfamic acid (dodecanoyl)-2,6-bis-(1-methylethyl)phenyl ester,
2,6-Bis(1-methylethyl)-N-[[[2,4,6-tris(1-methylethyl)phenyl]methyl]sulfonyl ]benzeneacetamide,
2,6-Bis(1-methylethyl)-N-[[[2,4,6-tris(1-methylethyl)phenyl]methyl]sulfonyl ]benzeneacetamide-sodium salt,
2,6-Bis(1-methylethyl)phenyl [[[2,4,6-tris(1-methylethyl)phenyl]methyl]sulfonyl ]carbamate,
2,6-Bis(1-methylethyl)phenyl[[[2,4,6-tris(1-methylethyl)phenyl]methyl]sulfonyl]carbamate-sodium salt,
Sulfamic acid (1-oxo-3,3-diphenylpropyl)-2,6-bis(1-methylethyl)phenyl ester,
Sulfamic acid [2,6-dichlorophenyl(acetyl)]-2,6-bis(1-methylethyl)phenyl ester,
Sulfamic acid [2,6-dichlorophenyl(acetyl)]-2,6-bis(1-methylethyl)phenyl ester,
Sulfamic acid trans-[(2-phenylcyclopropyl)-carbonyl]-2,6-bis(1-methylethyl)phenyl ester,
Sulfamic acid [2,5-dimethoxyphenyl(acetyl)]-2,6-bis(1-methylethyl)phenyl ester,
Sulfamic acid [2,4,6-trimethoxyphenyl(acetyl)]-2,6-bis(1-methylethyl)phenyl ester,
Sulfamic acid [2,4,6-trimethylphenyl(acetyl)]-2,6-bis(1-methylethyl)phenyl ester,
Sulfamic acid [2-thiophenyl(acetyl)]-2,6-bis(1-methylethyl)phenyl ester,
Sulfamic acid [3-thiophenyl(acetyl)]-2,6-bis(1-methylethyl)phenyl ester,
Sulfamic acid [2-methoxyphenyl(acetyl)]-2,6-bis(1-methylethyl)phenyl ester,
Sulfamic acid (oxophenylacetyl)-2,6-bis(1-methylethyl)phenyl ester,
Sulfamic acid [2-trifluoromethylphenyl(acetyl)]-2,6-bis(1-methylethyl)phenyl ester,
Sulfamic acid (1-oxo-2-phenylpropyl)-2,6-bis(1-methylethyl)phenyl ester,
Sulfamic acid (cyclopentylphenylacetyl)-2,6-bis(1-methylethyl)phenyl ester,
Sulfamic acid (cyclohexylacetyl)-2,6-bis(1-methylethyl)phenyl ester,
Sulfamic acid (diphenylacetyl)-2,6-bis(1-methylethyl)phenyl ester,
Sulfamic acid (triphenylacetyl)-2,6-bis(1-methylethyl)phenyl ester,
Sulfamic acid [(1-phenylcyclopentyl)carbonyl]-2,6-bis(1-methylethyl)phenyl ester,
Sulfamic acid (3-methyl-1-oxo-2-phenylpentyl)-2,6-bis(1-methylethyl)phenyl ester,
Sulfamic acid (1l-oxo-2-phenylbutyl)-2,6-bis(1-methylethyl)phenyl ester,
Sulfamic acid (cyclohexylphenylacetyl)-2,6-bis(1-methylethyl)phenyl ester,
Suifarnic acid (1-ox o-2,2-diphenylpropyl)-2,6-bis(1-methylethyl)phenyl ester,
Sulfamic acid [(9H-fluoren-9-yl)carbonyl]-2,6-bis(1-methylethyl)phenyl ester,
Sulfamic acid (1-oxo-3-phenylpropyl)-2,6-bis(1-methylethyl)phenyl ester,
Sulfamic acid [1-oxo-3-[2,4,6-tris(1-methylethyl)phenyl]-2-propenyl]-2,6-bis(1-methylethyl)phenyl ester,
Sulfamic acid [1-oxo-3-[2,4,6-tris(1-methylethyl)phenyl]propyl]-2,6-bis(1-methylethyl)phenyl ester,
Sulfamic acid [(acetyloxy)[2,4,6-tris(1-methylethyl)phenyl]acetyl]-2,6-bis(1-methylethyl)phenyl ester,
Sulfamic acid [hydroxy[2,4,6-tris(1-methylethyl)phenyl]acetyl]-2,6-bis(1-methylethyl)phenyl ester,
Sulfamic acid [fluoro[2,4,6-tris(1methylethyl)phenyl]acetyl]-2,6-bis(1-methylethyl)phenyl ester,
Sulfamic acid (3-methyl-1-oxo-2-phenylpentyl)-2,6-bis(1-methylethyl)phenyl ester sodium salt,
Sulfamic acid [[2,4,6-tris(1-methylethyl)phenoxy]acetyl]-2,6-bis(1-methylethyl)phenyl ester,
Sulfamic acid [[2,6-bis(1-methylethyl)phenoxy]acetyl]-2,6-bis(1-methylethyl)phenyl ester, and
Sulfamic acid [[2,4,6-tris(1-methylethyl)phenyl]acetyl]-2,6-bis(phenyl)phenyl ester.
   or a pharmaceutically acceptable salt, pro-drug or solvate thereof.

In therapeutic use as agents for treating of diabetes the compounds of Formulas (I) or pharmaceutically acceptable salts, pro-drugs or solvates thereof are administered to the patient at dosage levels of from 20 to 700 mg per day. For a normal human adult of approximately 70 kg of body weight, this translates into a dosage of from 0.3 to 10 mg/kg of body weight per day. The specific dosages employed, however, may be varied depending upon the requirements of the patient, the severity of the condition being treated, and the activity of the compound being employed. The determination of optimum dosages for a particular situation is within the skill of the art.

The compounds of Formula (I) may be administered in the form of a pro-drug which is broken down in the human or animal body to give a compound of the Formula (I). Examples of pro-drugs include in-vivo hydrolysable esters of a compound of the Formula (I). Various forms of pro-drugs are known in the art. For examples of such pro-drug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

An in-vivo hydrolysable ester of a compound of the Formula (I) containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆aLkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

An in-vivo hydrolysable ester of a compound of the Formula (I) containing a hydroxy group includes inorganic esters such as phosphate esters (including phosphoramidic cyclic esters) and (α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

A suitable pharmaceutically-acceptable salt of a compound of the invention is, for example, an acid-addition salt of a compound of the invention which is sufficiently basic, for example, an acid-addition salt with, for example, an inorganic or organic acid, for example ydrochloric, hydrobromic, sulphuric, phosphoric, trifluoroacetic, citric or maleic acid. In addition a suitable pharmaceutically-acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Compounds for use in the method of the invention can be prepared as described in International Patent Application, WO 94/26702, the contents of which are incorporated herein by reference.

The use of compounds of Formula (I) are provided as medicaments for the treatment of diabetes and/or obesity in a patient, eg, in men and/or women. To this end, a compound of Formula (I) can be provided as part of a pharmaceutical formulation which also includes a pharmaceutically acceptable diluent or carrier (eg, water). The formulation may be in the form of tablets, capsules, granules, powders, syrups, emulsions (eg, lipid emulsions), suppositories, ointments, creams, drops, suspensions (eg, aqueous or oily suspensions) or solutions (eg, aqueous or oily solutions). If desired, the formulation may include one or more additional substances independently selected from stabilising agents, wetting agents, emulsifying agents, buffers, lactose, sialic acid, magnesium stearate, terra alba, sucrose, corn starch, talc, gelatin, agar, pectin, peanut oil, olive oil, cacao butter and ethylene glycol.

The compound is preferably orally administered to a patient, but other routes of administration are possible, such as parenteral or rectal administration. For intravenous, subcutaneous or intramuscular administration, the patient may receive a daily dose of 0.1 mgkg⁻¹ to 30 mgkg⁻¹ (preferably, 5 mgkg^{31 1} to 20mgkg⁻¹) of the compound, the compound being administered 1 to 4 times per day. The intravenous, subcutaneous and intramuscular dose may be given by means of a bolus injection. Alternatively, the intravenous dose may be given by continuous infusion over a period of time. Alternatively, the patient may receive a daily oral dose which is approximately equivalent to the daily parenteral dose, the composition being administered 1 to 4 times per day. A suitable pharmaceutical formulation is one suitable for oral administration in unit dosage form, for example as a tablet or capsule, which contains between 10 mg and 1 g (preferably, 100 mg and 1 g) of the compound of the invention.

Buffers, pharmaceutically acceptable co-solvents (eg, polyethylene glycol, propylene glycol, glycerol or EtOH) or complexing agents such as hydroxy-propyl β cyclodextrin may be used to aid formulation.

The compounds described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets. For example in the treatment of diabetes mellitus pharmacotherapy may include the following main categories of treatment:
1) Insulin and insulin analogues;
2) Insulin secretagogues including sulphonylureas (for example glibenclamide, glipizide) and prandial glucose regulators (for example repaglinide, nateglinide);
3) Insulin sensitising agents including PPARg agonists (for example pioglitazone and rosiglitazone);
4) Agents that suppress hepatic glucose output (for example metformin).
5) Agents designed to reduce the absorption of glucose from the intestine (for example acarbose);
6) Agents designed to treat the complications of prolonged hyperglycaemia;
7) Anti-obesity agents (for example sibutramine and orlistat);
8) Anti- dyslipidaemia agents such as, HMG-CoA reductase inhibitors (statins, eg pravastatin); PPARα agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
9) Antihypertensive agents such as, β blockers (eg atenolol, inderal); ACE inhibitors (eg lisinopril); Calcium antagonists (eg. nifedipine); Angiotensin receptor antagonists (eg candesartan), β antagonists and diuretic agents (eg. furosemide, benzthiazide);
10) Haemostasis modulators such as, antithrombotics, activators of fibrinolysis and antiplatelet agents; thrombin antagonists; factor Xa inhibitors; factor VIla inhibitors); antiplatelet agents (eg. aspirin, clopidogrel); anticoagulants (heparin and Low molecular weight analogues, hirudin) and warfarin; and
11) Anti-inflammatory agents, such as non-steroidal anti-infammatory drugs (eg. aspirin) and steroidal anti-inflammatory agents (eg. cortisone).
   Assays
   ACAT

The ability of compounds to inhibit ACAT can be measured using an in-vitro test described in Field & Salone (1982) Biochemica et Biophysica, 712, 557-570. The test assesses the ability of a test compound to inhibit the acylation of cholesterol by oleic acid by measuring the amount of radiolabeled cholesterol oleate formed from radiolabeled oleic acid in a tissue preparation containing rat liver microsomes.

DGAT

The ability of compounds to inhibit DGAT can be measured as described in Coleman (1992) Methods in Enzymology 209, 98-102.

### EXAMPLE 1

The inhibitory activity of sulfamic acid [[2,4,6-tris(1-methylethyl)phenyl]acetyl-2,6-bis(1-methylethyl)phenyl ester against DGAT1, DGAT2 and ACAT was measured in rat and human liver microsomes. DGAT1 and DGAT2 can be distinguished since DGAT2 is not active at high magnesium concentrations (concentrations of 50 mM or higher) whilst DGAT1 retains its activity at high magnesium concentrations.

The results obtained, expressed as the concentration of inhibitor at which 50% of the enzyme activity is inhibited, i.e. the IC₅₀, are as follows:

Rat liver microsomes

| | IC₅₀ (μM) |
|---|---|
| DGAT1 and DGAT2 | 3.41 |
| DGAT1(100 mM Mg) | 2.28 |
| ACAT | 1.03 |

Human liver microsomes

| | IC₅₀ (μM) |
|---|---|
| DGAT1 and DGAT2 | 7.95 |
| DGAT1(100 mM Mg) | 3.03 |
| ACAT | 5.67 |

1. The use of a compound of Formula (I): 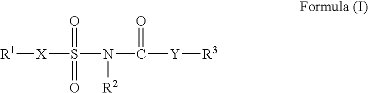
wherein:
X and Y are independently selected from: oxygen, sulphur and (—CR^{a}R^{b}—)ₙ; wherein:
n is an integer of from 1 to 4 and
R^{a} and R^{b} are each independently selected from hydrogen, C₁₋₆alkyl, C₁₋₆alkoxy, halo, hydroxy, C₁₋₆alkanoyloxy, C₃₋₁₂cycloalkyl and optionally substituted phenyl or R^{a} and R^{b}together form a C₅₋₁₂spirocycloalkyl or a carbonyl;
with the proviso that at least one of X and Y is (—CR^{a}R^{b}—)ₙ and with the further proviso that when X and Y are both (—CR^{a}R^{b}—)ₙ and R^{a} and R^{b} are hydrogen and n is 1, then R¹ and R³ are both aryl;
R² is hydrogen, a C₁₋₈alkyl or benzyl;
R¹ and R³ are independently selected from
(a) phenyl or phenoxy wherein the phenyl or phenoxy group is optionally substituted with 1 to 5 substituents independently selected from phenyl, C₁₋₆alkyl, C₁₋₆alkoxy, phenoxy, hydroxy, fluorine, chlorine, bromine, nitro, trifluoromethyl, carboxy, C₁₋₄ alkoxycarbony and —(CH₂)ₚNR₄R₅ wherein p is 0 or 1, and R⁴ and R⁵ are independently selected from hydrogen and C₁₋₄alkyl;
(b) naphth-1-yl or naphth-2-yl wherein the naphthyl group is optionall substituted with from 1 to 3 substituents independently selected from phenyl, C₁₋₆alkyl, C₁₋₆alkoxy, phenoxy, hydroxy, fluorine, chlorine, bromine, nitro, trifluoromethyl, carboxy, C₁₋₄alkoxycarbony and —(CH₂)ₚNR₄R₅ wherein p, R⁴ and R⁵ are as defined above;
(c) arylC₁₋₆alkyl;
(d) C₁₋₂₀alkyl or C₁₋₂₀alkenyl; and
(e) adamantyl or a C₃₋₁₂cycloalkyl;
or a pharmaceutically acceptable salt, pro-drug or solvate thereof in the manufacture of a medicament for the treatment of diabetes and/or obesity.

2. The use according to claim 1 wherein R¹ is phenyl.

3. The use according to claim 2 wherein R¹ is phenyl disubstituted in the 2,6-positions.

4. The use according to any one of claims 1,2 or 3 wherein R³ is phenyl.

5. The use according to claim 4 wherein R³ is phenyl disubstituted in the 2,6-positions.

6. The use according to claim 1 wherein R¹ is phenyl disubstituted in the 2,6-positions and R³ is phenyl trisubstituted in the 2,4,6-positions.

7. The use according to claim 1 wherein R¹ is 2,6-bis(1-methylethyl)phenyl and R³ is 2,6-bis(1-methylethyl)phenyl or 2,4,6-tris(1-methylethyl)phenyl.

8. The use according to claim 1 wherein:
one of R¹ and R³ is the group 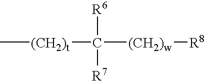
wherein
t is 0 to 4;
w is 0 to 4 with the proviso that the sum of t and w is not greater than 5;
R⁶ and R⁷ are independently selected from hydrogen or C₁₋₆alkyl, or when R⁶ is hydrogen, R⁷ can be selected from the groups defined for R⁸; and R⁸ is phenyl optionally substituted with from 1 to 3 substituents selected C₁₋₆allyl C₁₋₆alkoxy, phenoxy, hydroxy, fluorine, chlorine, bromine, nitro, trifluoromethyl, carboxy, C₁₋₄alkoxycarbonyl, or —(CH₂)ₚNR⁴R⁵ wherein p, R⁴ and R⁵ are as defined above.

9. The use according to claim 1 wherein
X is oxygen;
Y is (CR^{a}R^{b})ₙ wherein
n is an integer of from 1 to 4 and
R^{a} and R^{b} are each independently hydrogen, C₁₋₆alkyl, optionally substituted phenyl, halo, hydroxy, C₁₋₆alkoxy, C₁₋₆alkanoyloxy, cycloalkyl, or R^{a} and R^{b} taken together form a carbonyl or C₃₋₁₀spirocycloalkyl;
R¹ is selected from optionally substituted phenyl, C₁₋₆alkyl or C₃₋₁₀cycloalkyl;
R² is hydrogen;
R³ is selected from optionally substituted phenyl, C₁₋₁₀alkyl, C₃₋₈cycloalkyl and optionally substituted phenoxy.

10. The use according to claim 1 wherein
X is oxygen;
Y is (CR^{a}R^{b})ₙ wherein n is an integer of from 1 to 2;
R¹ is optionally substituted phenyl;
R² is hydrogen;
R³ is selected from optionally substituted phenyl, optionally substituted phenoxy, C₁₋₁₀alkyl, and C₃₋₁₀cycloalkyl;
R^{a} and R^{b} are independently selected from hydrogen, C₁₋₆alkyl, optionally substituted phenyl, halogen, hydroxy, C₁₋₆alkoxy, C₁₋₆alkanoyloxy, cycloalkyl, or R^{a} and R^{b} taken together form a carbonyl or a spirocycloalkyl.

11. The use according to claim 1 wherein
X is oxygen;
Y is (—CR^{a}R^{b}—)n wherein n is an integer of from 1 to 4 and R′ and R″ are each independently hydrogen, alkyl, alkoxy, halogen, hydroxy, acyloxy, cycloalkyl, phenyl optionally substituted or R′ and R″ together form a spirocycloalkyl or a carbonyl;
R¹ and R³ are independently selected from
(a) phenyl or phenoxy wherein the phenyl or phenoxy group is optionally substituted with 1 to 5 substituents independently selected from phenyl, C₁₋₆alkyl, C₁₋₆alkoxy, phenoxy, hydroxy, fluorine, chlorine, bromine, nitro, trifluoromethyl, carboxy, C₁₋₄alkoxycarbony and —(CH₂)ₚNR₄R₅ wherein p is 0 or 1, and R⁴ and R⁵ are independently selected from hydrogen or C₁₋₄alkyl;
(b) naphth-1-yl or naphth-2-yl wherein the naphthyl group is optionally substituted with from 1 to 3 substituents independently selected from phenyl, C₁₋₆-alkyl, C₁₋₆alkoxy, phenoxy, hydroxy, fluorine, chlorine, bromine, nitro, trifluoromethyl, carboxy, C₁₋₄alkoxycarbony and—(CH₂)ₚNR₄R₅ wherein p, R⁴ and R⁵ are as defined above;
(c) arylC₁₋₆zalkyl;
(d) C₁₋₂₀alkyl or C₁₋₂₀oalkenyl; and
(e) adamantyl or a C₃₋₁₂cycloalkyl
R² is hydrogen, a C₁₋₈alkyl or benzyl;

12. The use according to claim 1 wherein the compound is selected from:
Sulfamic acid (phenylacetyl)-2,6-bis(1-methylethyl)phenyl ester,
Sulfamic acid[[2,6-bis(1-methylethyl)phenyl]acetyl]-2,6-bis(1-methylethyl)phenyl ester,
Sulfamic acid [[2,4,6-tris(1-methylethyl)phenyl]acetyl-2,4,6-tris(1-methylethyl)phenyl ester,
Sulfamic acid[[2,6-bis(1-methylethyl)phenyl]acetyl]-2,4,6-tris(1-methylethyl)phenyl ester,
Sulfamic acid[adamantaneacetyl]-2,6-bis[1-methylethyl)phenyl ester,
Sulfamic acid[[2,6-bis(1-methylethyl)phenyl]acetyl]-2,6-bis(1-methylethyl)phenyl ester-sodium salt,
Sulfamic acid[[2,4,6-tris(1-methylethyl)phenyl]acetyl]-2,6-bis(1-methylethyl)phenyl ester-sodium salt,
Sulfamic acid (decanoyl)-2,6-bis-(1-methylethyl)phenyl ester,
Sulfamic acid (dodecanoyl)-2,6-bis-(1-methylethyl)phenyl ester,
2,6-Bis(1-methylethyl)-N-[[[2,4,6-tris(1-methylethyl)phenyl]methyl]sulfonyl] benzeneacetamide,
2,6-Bis(1-methylethyl)-N-[[[2,4,6-tris(1-methylethyl)phenyl]methyl]sulfonyl] benzeneacetamide-sodium salt.
2,6-Bis(1-methylethyl)phenyl[[[2,4,6-tris(1-methylethyl)phenyl]methyl]sulfonyl]carbamate,
2,6-Bis(1-methylethyl)phenyl[[[2,4,6-tris(1-methylethyl)phenyl]methyl]sulfonyl]carbamate-sodium salt,
Sulfamic acid (1-oxo-3,3-diphenylpropyl)-2,6-bis(1-methylethyl)phenyl ester,
Sulfamic acid [2,6-dichlorophenyl(acetyl)]-2,6-bis(1-methylethyl)phenyl ester,
Sulfamic acid [2,6-dichlorophenyl(acetyl)]-2,6-bis(1-methylethyl)phenyl ester,
Sulfamic acid trans-[(2-phenylcyclopropyl)carbonyl]-2,6-bis(1-methylethyl)phenyl ester,
Sulfamic acid [2,5-dimethoxyphenyl(acetyl)]-2,6-bis(1-methylethyl)phenyl ester,
Sulfamic acid [2,4,6-trimethoxyphenyl(acetyl)]-2,6-bis(1-methylethyl)phenyl ester,
Sulfamic acid [2,4,6-trimethylphenyl(acetyl)]-2,6-bis(1-methylethyl)phenyl ester,
Sulfamic acid [2-thiophenyl(acetyl)]-2,6-bis(1-methylethyl)phenyl ester,
Sulfamic acid [3-thiophenyl(acetyl)]-2,6-bis(1-methylethyl)phenyl ester,
Sulfamic acid [2-methoxyphenyl(acetyl)]-2,6-bis(1-methylethyl)phenyl ester,
Sulfamic acid (oxophenylacetyl)-2,6-bis(1-methylethyl)phenyl ester,
Sulfamic acid [2-trifluoromethylphenyl(acetyl)]-2,6-bis(1-methylethyl)phenyl ester,
Sulfamic acid (1-oxo-2-phenylpropyl)-2,6-bis(1-methylethyl)phenyl ester,
Sulfamic acid (cyclopentylphenylacetyl)-2,6-bis(1-methylethyl)phenyl ester,
Sulfamic acid (cyclohexylacetyl)-2,6-bis(1-methylethyl)phenyl ester,
Sulfamic acid (diphenylacetyl)-2,6-bis(1-methylethyl)phenyl ester,
Sulfamic acid (triphenylacetyl)-2,6-bis(1-methylethyl)phenyl ester,
Sulfamic acid [(11-phenylcyclopentyl)carbonyl]-2,6-bis(1-methylethyl)phenyl ester,
Sulfamic acid (3-methyl-1-oxo-2-phenylpentyl)-2,6-bis(1-methylethyl)phenyl ester,
Sulfamic acid (1-oxo-2-phenylbutyl)-2,6-bis(1-methylethyl)phenyl ester,
Sulfamic acid (cyclohexylphenylacetyl)-2,6-bis(1-methylethyl)phenyl ester,
Sulfamic acid (1-oxo-2,2-diphenylpropyl)-2,6-bis(1-methylethyl)phenyl ester,
Sulfamic acid [(9H-fluoren-9-yl)carbonyl]-2,6-bis(1-methylethyl)phenyl ester,
Sulfamic acid (1-oxo-3-phenylpropyl)-2,6-bis(1-methylethyl)phenyl ester,
Sulfamic acid [1-oxo-3-[2,4,6-tris(1-methylethyl)phenyl-2-propenyl]-2,6-bis(1-methylethyl)phenyl ester,
Sulfamic acid [1-oxo-3-[2,4,6-tris(1-methylethyl)phenyl]propyl]-2,6-bis(1-methylethyl)phenyl ester,
Sulfamic acid [(acetyloxy)[2,4,6-tris(1-methylethyl)phenyl]acetyl]-2,6-bis(1-methylethyl )phenyl ester,
Sulfamic acid [hydroxy[2,4,6-tris(1-methylethyl)phenyl]acetyl]-2,6-bis(1-methylethyl)phenyl ester,
Sulfamic acid [fluoro[2,4,6-tris(1-methylethyl)phenyl]acetyl]-2,6-bis(1-methylethyl)phenyl ester,
Sulfamic acid (3-methyl-1-oxo-2-phenylpentyl)-2,6-bis(1-methylethyl)phenyl ester sodium salt,
Sulfamic acid [[2,4,6-tris(1-methylethyl)phenoxy]acetyl]-2,6-bis(1-methylethyl)phenyl ester,
Sulfamic acid [[2,6-bis(1-methylethyl)phenoxy]acetyl]-2,6-bis(1-methylethyl)phenyl ester, and
Sulfamic acid [[2,4,6-tris(1-methylethyl)phenyl]acetyl]-2,6-bis(phenyl)phenyl ester.
or pharmaceutically acceptable salt, pro-drug or solvate.

13. The use according to claim 1 wherein the compound is:
sulfamic acid[[2,4,6-tris(1-methylethyl)phenyl]acetyl-2,6-bis(1-methylethyl)phenyl ester.
or pharmaceutically acceptable salt, pro-drug or solvate.

14. The use according to any one of the preceding claims wherein the use is the manufacture of a medicament for the treatment of type II diabetes.

15. The use according to any one of the preceding claims wherein the use is the manufacture of a medicament for the treatment of obesity.

16. The use according to any one of the preceding claims wherein the use is the manufacture of a medicament for the treatment of insulin resistance.

17. The use according to any one of the preceding claims wherein the use is the manufacture of a medicament for the treatment of impaired glucose tolerance.
